(19) United States
(12) Patent Application Publication
(10) Pub. No.: US 2014/0091185 A1
(43) Pub. Date: Apr. 3, 2014

(54) FIELD POST

(71) Applicant: FLEXBAR MACHINE CORP., Islandia, NY (US)

(72) Inventors: Steven Culver, Lake Ronkonkoma, NY (US); Donohue Michael, Ronkonkoma, NY (US)

(73) Assignee: Flexbar Machine Corp., Islandia, NY (US)

(21) Appl. No.: 13/630,362

(22) Filed: Sep. 28, 2012

(51) Int. Cl. A61B 19/00; F16M 13/02

(52) U.S. Cl. 248/214

(57) ABSTRACT

An adjustable height field post includes an inner shaft and an outer tube configured to receive and slidably engage the inner shaft. A clamp body is provided at one end of the inner shaft such that the field post can be securely attached to an operating table. An adjustment mechanism is incorporated into the inner shaft and the outer tube such that the height of the field post can be adjusted to accommodate various working conditions and also prevent any portion of the same from extending beyond an upper most attached post coupling.

### BACKGROUND

1. Technical Field

The present principles relate to surgical procedures. More particularly, they relate to an adjustable height operating room field post for holding and securing various surgical devices and aids used in performing surgical procedures.

2. Related Art

The operating room environment can be difficult to navigate and particularly as it relates to the performing of particular procedures where retraction assemblies or other devices are required. By way of example, in order secure a retraction system to a surgical site, field posts are used.

A field post is securely attached to the operating table and one or more post couplings can be attached to the same. The post couplings allow the attachment of additional posts or arms to hold various surgical apparatuses, such as, for example, retractors, surgical holders and positioners, etc. Since the size of the patient changes from procedure to procedure, often times the field post is longer than needed. In these instances, the connected assemblies are positioned lower down on the field post, thus leaving an upper portion of the field post that protrudes upward beyond the upper most connected post coupling. The remaining portion of the field post clutters the surgical field and can be an obstacle to the doctor and/or assistants involved in performing the surgical procedure. In some instances, this remaining portion of the field post can potentially result in a harmful disruption to the surgical field and/or procedure (e.g., surgeon or assistants can bump into the top of the field post during the surgical procedure).

There is thus a need for an improved field post which eliminates the aforementioned problems with existing operating room field posts.

### SUMMARY

According to an implementation, the adjustable height field post includes an inner shaft having two opposing ends, an outer tube configured to co-axially receive a first end the inner shaft, a height adjustment mechanism configured into the inner shaft and outer tube to allow height adjustment of the field post, and a field post head connected to a second end of the inner shaft, said field post head releasably connecting the field post to a rail or bar.

According to another implementation, the adjustable height field post includes a telescoping shaft, a height adjustment mechanism configured into the telescoping shaft to allow for height adjustment and the locking of the telescoping shaft in a predetermined height position, and a field post head connected to an end of the telescoping shaft and configured for releasably connecting the field post to a rail or bar.

These and other aspects, features and advantages of the present principles will become apparent from the following detailed description of exemplary embodiments, which is to be read in connection with the accompanying drawings.

### BRIEF DESCRIPTION OF THE DRAWINGS

The present principles may be better understood in accordance with the following exemplary figures, in which:

FIG. 1a is view of the field post with the clamp fully backed out, according to an implementation of the present principles;

FIG. 1b is a view of the field post with the clamp in a locked positioned, according to an implementation of the present principles;

FIG. 1c is a view of the field post extended, according to an implementation of the present principles;

FIG. 2 is an exploded perspective view of the field post, according to an implementation of the present principles;

FIGS. 3a and 3b are views of the inner shaft of the field post, according to an implementation of the present principles;

FIG. 4a is a side view of the handle used to connect and disconnect the field post to an desired rail or bar;

FIG. 4b is an exploded view of the field post head, according to an implementation of the present principles;

FIGS. 5a and 5b show the details of the rotation joint of the field post, according to an implementation of the present principles; and

FIG. 6 is a side by side view of a field post of the prior art and the field post of the present principles.

### DETAILED DESCRIPTION

The present principles are directed to field posts for use in an operating room environment. More specifically, it relates to adjustable height field posts for use in an operating room environment.

The present description illustrates the present principles. It will thus be appreciated that those skilled in the art will be able to devise various arrangements that, although not explicitly described or shown herein, embody the present principles and are included within its spirit and scope.

All examples and conditional language recited herein are intended for pedagogical purposes to aid the reader in understanding the present principles and the concepts contributed by the inventor(s) to furthering the art, and are to be construed as being without limitation to such specifically recited examples and conditions.

Moreover, all statements herein reciting principles, aspects, and embodiments of the present principles, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

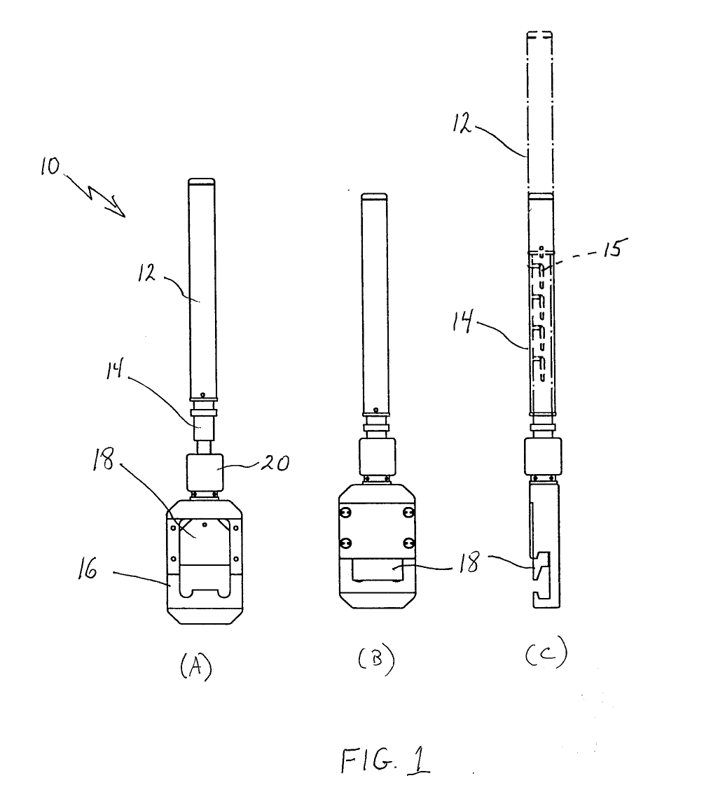

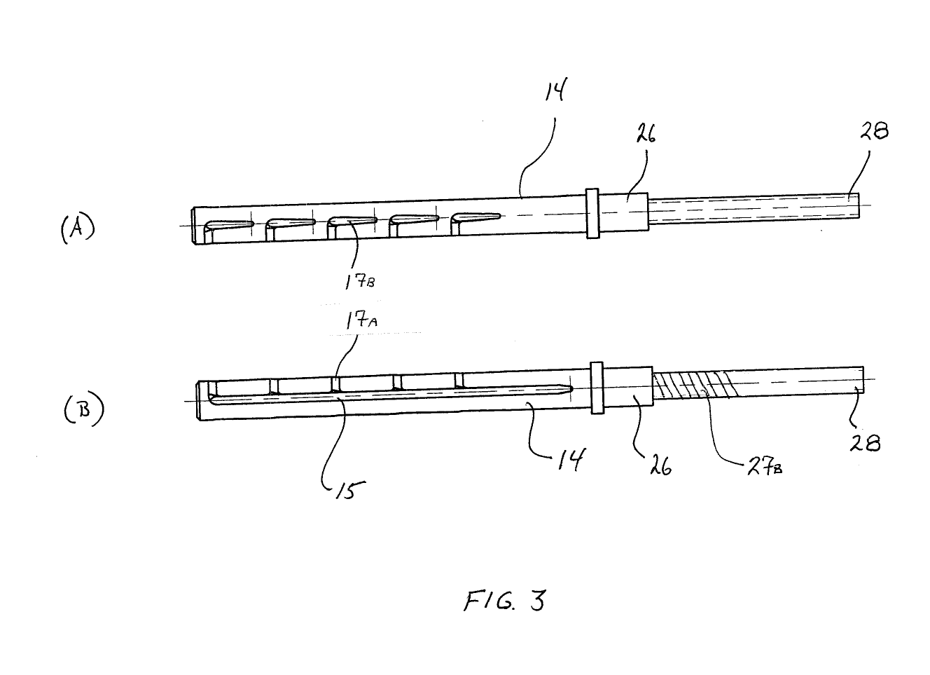

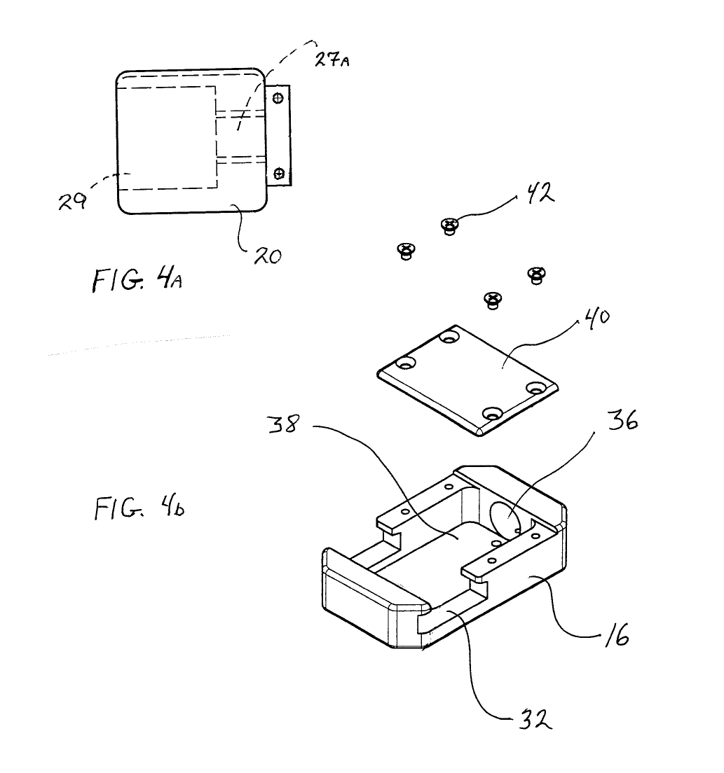

Referring to FIG. 1, there is shown the adjustable height field post 10 according to an embodiment of the present principles. The field post 10 has an outer tube 12, and inner shaft 14 and a field post head 16 that receives an end of the inner shaft 14 and includes a clamp body 18. FIG. 1a shows the field post 10 with the clamp 18 full backed out. FIG. 1b shows the field post 10 with the clamp 18 engaged (as if it were attached to a rail or bar which may, for example, be part of the operating table), and FIG. 1c shows the field post 10 with the outer tube 12 extended and the slots 15 of the inner shaft 14 shown in phantom. As will be shown below in reference to FIGS. 3 and 4a, by rotating the handle 20, the clamp 18 moves up and down and engages on the rail or bar which may, for example, be part of the operating table.

In use, the outer tube 12 of the field post is configured such that there are no sharp edges, and the tube can slidably move freely over inner shaft 14 and can be locked in place using the slots 15 in the inner shaft. The inner shaft 14 and outer tube 12 may be made of any suitable material. Examples of such materials include Aluminum, Stainless Steel, Plastic and Titanium.

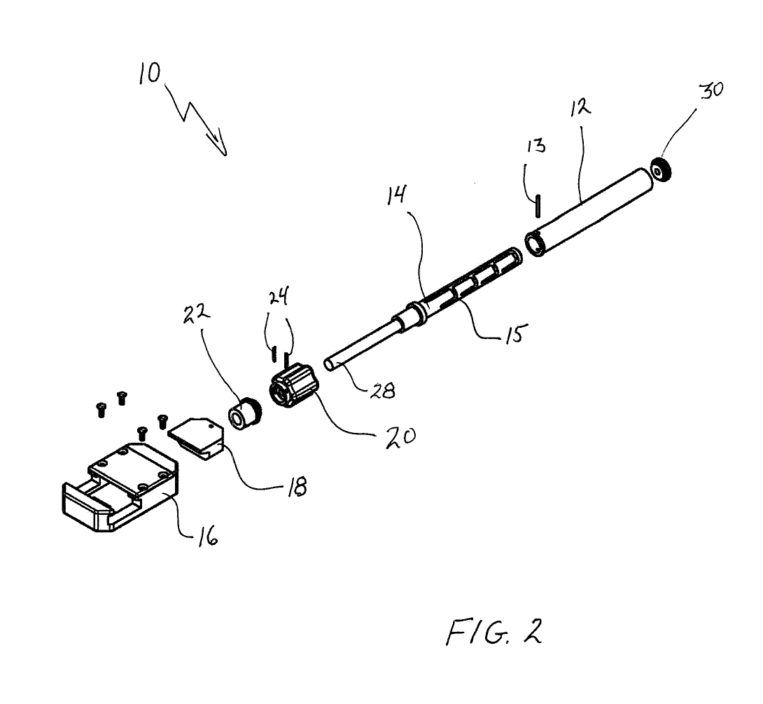

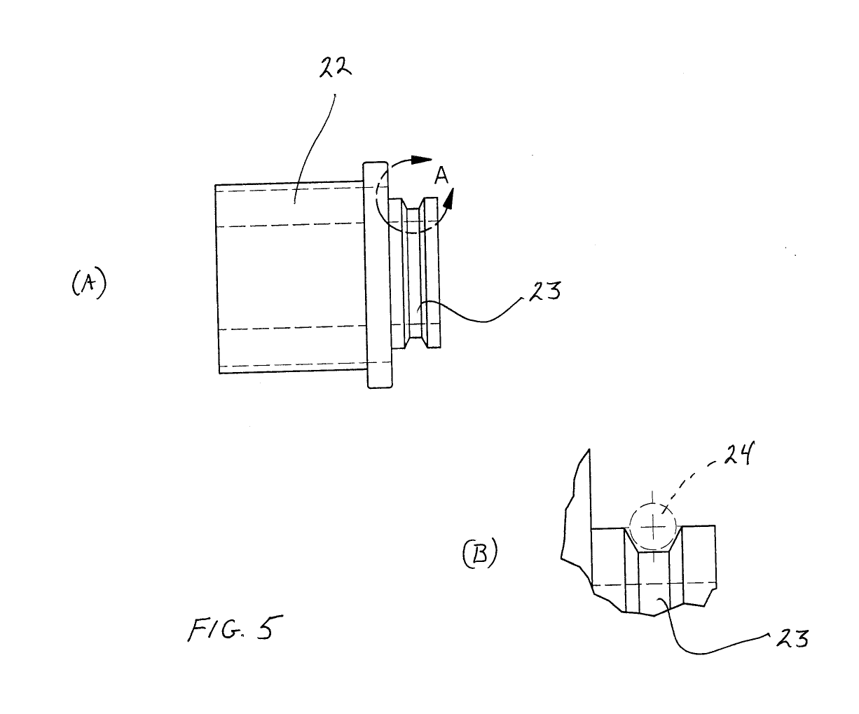

FIG. 2 shows an exploded view of the field post 10 according to an implementation of the present principles. As shown, the outer tube 12 is configured to fit over the inner shaft 14 and includes a pin 13 that is used to adjust the height of the field post. A cap 30 encloses the top exposed end of the outer tube 12. Inner shaft 14 includes the grooves or slots 15 that are engaged by pin 13 and together provide the height adjustment system or means of the field post. The details of slots 15 will be described in more detail below with reference to FIG. 3. One end 28 of the inner shaft 14 is configured to be attached to the clamp body 18 and receives the handle 20 and corresponding joint 22. The field post head 16 encloses the clamp 18 and is configured to attach the field post 10 to a rail or bar. In the preferred implementation, joint 22 is fixed onto clamp body field post head 16, and handle 20 is pinned to joint 22 using pins 24 (See FIGS. 5a and 5b) which allows for rotation between the handle 20 and the joint 22. The inner shaft 14 is threaded through handle 20 and pinned to clamp body 18 (i.e., after clamp body 18 is disposed within field end post 16). Rotation of handle 20 allows for some axial movement of inner shaft 14 which (as shown in FIG. 1a) provides for a backing out of clamp body 18 so the field end post 16 can be attached to the desired rail or bar.

Referring to FIG. 3, there is shown the inner shaft 14 with a longitudinal slot 15 and transverse and height adjustment slots 17a and 17b, respectively. In operation, the outer tube 12 (not shown) receives the inner shaft 14 and the pin 13 is inserted through the outer tube such that it passes through the longitudinal slot 15 (on both sides of the inner shaft). As will be evident to those of skill in the art, once the outer tube 12 has been positioned such that pin 13 is completely inserted and engaged in slot 15, the outer tube 12 will move freely and axially along the same. The transverse slots 17a are positioned at predetermined locations along longitudinal slot 15. Additional longitudinal slots 17b of predetermined length, and connected to transverse slots 17a provide the height adjustment by receiving the pin 13 and locking in the same at the base of the respective slot 17b.

As will be apparent from the above description, when the field post is mounted on a rail or bar (via field post head 16), the user may then slide outer tube 12 along slot 15 to determine the desired height. To do this, the user pulls up on the outer tube and turns the same counter-clockwise after pulling up. This will release the outer tube from the height adjustment slot 17b it is currently in. When the desired height is reached, the user will rotate the outer tube 12 in a clockwise direction (into the corresponding transverse slot 17a) and lower it into the corresponding height adjustment slot 17b. Although the exemplary embodiment of FIG. 3 shows five (5) height adjustment slots 17b, more or less could be implemented without departing from the spirit of the invention. The shoulder or handle connection 26 integrated with the inner shaft with the handle 20 and includes the shaft 28 which provides for connection to the clamp 18 within the field post head 16. The shaft 28 preferably includes threading 27b which engages threading 27 within the handle 20.

In other contemplated implementations, the height adjustment of the outer tube 12 with respect to the inner shaft 14 can be performed using a lead screw, a spring loaded button/pin and/or a rack/pinion combination.

FIG. 4a shows the handle 20 according to an implementation of the present principles. Handle 20 has a larger opening 29 configured to receive the shoulder 26. The smaller opening 27a receives the shaft 28 and has a mating thread engagement with the threads 27b disposed thereon. As described above, it is the rotation of the handle 20, working in conjunction with the threaded engagement between the shaft and the handle, that allows for the backing out of the clamp 18 and the subsequent securing of the same to the desired rail or bar.

FIG. 4b shows the field post head 16 according to an implementation of the present principles. The field post head 16 includes a hole 36 configured to receive the end 28 of the inner tube 14 and which attaches to the clamp 18 (not shown). The receiving area 38 within the field post head 16 is configured to receive the clamp body 18. A cover 40 and screws 42 protects the clamp body 18 when positioned within the field post head accordingly. Field post head 16 includes a slot 32 which is configured to receive the rail or other means for connecting the same to the operating room table. Although in the example show the slot 32 is substantially rectangular to receive a rectangular portion of the operating table, with the corresponding clamp body 18 is configured accordingly, it will be appreciated that slot 32 (and corresponding clamp 18) can be made in other shapes (e.g., round, triangular, etc.) to receive and secure the field post to a round or angularly shaped rail on the operating table.

FIGS. 5a and 5b show a close up view of the rotation joint 22 according to an implementation of the present principles. As shown a circumferential groove 23 is provided at the end which engages the rotation handle 24. Once the handle 20 is positioned on the rotation joint, the pins 24 are inserted through the handle such that the same fall within the groove 23 and not only operate to prevent the handle from being separated from the rotation joint, but also enables the same to be rotated around the groove. In this manner, the rotation handle 20 (and its threaded engagement with shaft 28 of inner shaft 14) causes the inner shaft 14 to be axially displaced. As described above, this moves the clamp body 18 to engage or disengage the rail or bar to which it is to be affixed.

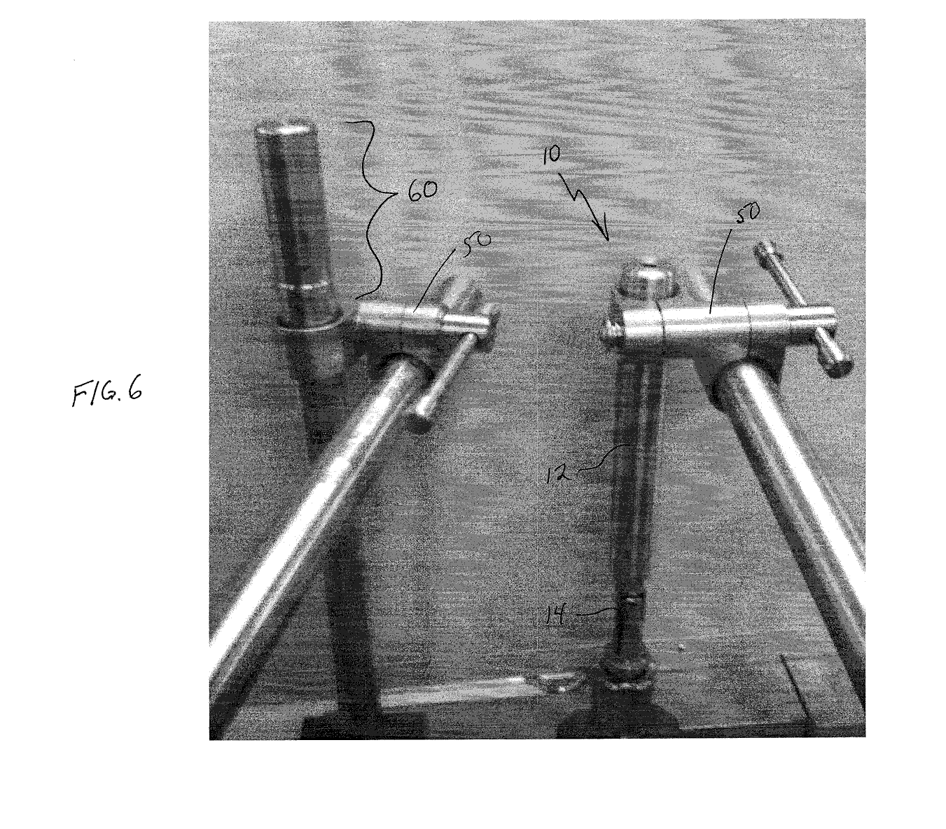

FIG. 6 shows a side by side comparison of a field post of the prior art compared to the field post 10 of the present invention. As shown, a post coupling 50 is positioned at the same height, yet the field post of the prior art has a larger portion 60 that remains above the post coupling. It is this extra portion 60 that clutters the surgical field and can be an obstacle to the doctor and/or assistants involved in performing the surgical procedure. The field post 10 of the present invention overcomes the problems presented by the extra portion 60 by allowing the entire post to be adjustable in height such that an upper most post coupling 50 is always positioned from the top thereof and can be lowered as required.

Although the illustrative embodiments have been described herein with reference to the accompanying drawings, it is to be understood that the present principles is not limited to those precise embodiments, and that various changes and modifications may be effected therein by one of ordinary skill in the pertinent art without departing from the scope or spirit of the present principles. All such changes and modifications are intended to be included within the scope of the present principles as set forth in the appended claims

What is claimed is:

1. An adjustable height field post comprising:
an inner shaft having two opposing ends;
an outer tube configured to co-axially receive a first end the inner shaft;
a height adjustment mechanism configured into the inner shaft and outer tube to allow height adjustment of the field post; and
a field post head connected to a second end of the inner shaft, said field post head releasably connecting the field post to a rail or bar.

2. The adjustable height field post according to claim 1, wherein said height adjustment mechanism comprises:
a plurality of slots integrated into the inner shaft; and
a pin secured by the outer tube, said pin selectively engaging said plurality of slots to provide a secured displacement of the inner shaft with respect to the outer tube and thereby the height adjustment of the field post.

3. The adjustable height field post according to claim 1, wherein said field post head comprises
an aperture configured to receive the second end of the inner shaft;
a clamp body positioned within a receiving area and attached to the second end of the inner shaft;
a cover for securing the clamp body within the receiving area;
means for engaging a rail or bar; wherein said clamp body and said engaging means cooperate to secure the field post head and thereby the field post to the rail or bar.

4. The adjustable field post according to claim 2, wherein said plurality of slots comprises:
a pair of diametrically opposed longitudinal slots integrated into the inner shaft;
a plurality of transverse slots in communication with the longitudinal slots; and
a plurality of height adjustment slots in communication with the transverse slots.

5. The adjustable height field post according to claim 4, wherein said plurality of transverse slots and plurality of finite length longitudinal slots are spaced from each other by a predetermined amount to provide a variable range of height adjustment in a single field post.

6. The adjustable height field post according to claim 1, further comprising a rotation means integrated into the inner shaft and configured to enable the inner shaft to be axially displaced for releasable attachment to a rail or bar via the field post head.

7. The adjustable height field post according to claim 1, wherein the field post is collapsible via said height adjustment mechanism.

8. An adjustable height field post comprising:
a telescoping shaft;
a height adjustment mechanism configured into the telescoping shaft to allow for height adjustment and the locking of the telescoping shaft in a predetermined height position; and
a field post head connected to an end of the telescoping shaft and configured for releasably connecting the field post to a rail or bar.

9. The adjustable height field post according to claim 8, wherein said telescoping shaft comprises:
an inner shaft having two opposing ends;
an outer tube configured to co-axially receive a first end the inner shaft;

10. The adjustable height field post according to claim 8, wherein said field post head comprises
an aperture configured to receive the end of the telescoping shaft;
a clamp body positioned within a receiving area and attached to the end of the telescoping shaft;
a cover for securing the clamp body within the receiving area; and
means for engaging a rail or bar; wherein said clamp body and said engaging means cooperate to secure the field post head and thereby the field post to the rail or bar.

11. The adjustable height field post according to claim 10, wherein said engaging means comprises a threaded handle and a mating threaded portion of the telescoping shaft, wherein rotation of the threaded handle causes axial displacement of the end of the telescoping shaft and thereby the connected clamp body, said axial displacement enabling the releasable connection of the field end post to a desired rail or bar.
